(19) United States
(12) Patent Application Publication
(10) Pub. No.: US 2005/0067951 A1
(43) Pub. Date: Mar. 31, 2005

(54) Triarylamine derivatives and their use in organic electroluminescent and electrophotographic devices

(75) Inventors: Andreas M. Richter, Plossmitz (DE); Volker Lischewski, Wolfen (DE)

Correspondence Address: FOLEY & LARDNER, 777 EAST WISCONSIN AVENUE, SUITE 3800, MILWAUKEE, WI, 53202-5308, US

(73) Assignee: Sensient Imaging Technologies GmbH

(21) Appl. No.: 10/899,522

(22) Filed: Jul. 27, 2004

Related U.S. Application Data
(63) Continuation of application No. PCT/DE02/04758, filed on Dec. 19, 2002.

(30) Foreign Application Priority Data
Jan. 28, 2002 (DE) DE 102 03 328.5

(51) Int. Cl. H05B 33/12; C07C 211/43; C07D 29/82; C07D 37/91; C07D 333/76; C07F 7/10

(52) U.S. Cl. 313/504; 428/690; 428/917; 313/506; 564/426; 564/427; 564/428; 564/434; 548/442; 549/43; 549/460; 556/406

(57) ABSTRACT

New triarylamine derivatives containing special space-filling wing groups and to the use thereof as a hole transport material in electrographic and electrolumkinescent devices are provided. In the triarylamine derivatives, n-1-10, R¹—R⁴ represent optionally substituted phenyl, biphenylyl, naphthyl, phenanthrenyl, anthracenyl, fluorenyl, triarylmethyl aryl, or triarylsilyl aryl; Ar represents a biphenylene, triphenylene, tetraphenylene or fluorenylene-type bridge.

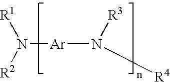

### CROSS-REFERENCE TO RELATED PATENT APPLICATIONS

This application claims priority of International Patent Application PCT/DE02/04758, filed Dec. 19, 2002, and German Patent Application DE 102030328.5, filed Jan. 28, 2002, the disclosures of which are incorporated herein by reference in their entirety.

### BACKGROUND

This application relates to new triarylamine derivatives provided with special space-filling wing groups, and their use as hole transfer material in electrophotographic and electroluminescent devices.

Electrophotographic and electroluminescent devices and the use of triarylamine derivatives, such as triarylamine dimers and triarylamine tetramers, have been known for a long time.

Currently, tris(-8-hydroxychinolino)-aluminium is used as preferred luminescent material, the electroluminescence of which has been known since 1965. This metal chelate complex, which, in some cases, can be doped with coumarin, luminesces in a green colour, and the metal used can also be beryllium or gallium.

Although in the beginning a relatively high turn-on voltage of more than 10 volts was required, the necessary voltage was able to be reduced to less than 10 volts by arranging an additional hole transport layer between the anode and the luminescent layer.

Apart from phthalocyanines and biphenylyl oxadiazol derivatives, preferred hole transfer materials used are N,N′-diphenyl-N,N′-bis(m-tolyl)-benzidine (TPD) and N,N′-diphenyl-N,N′-di-naphth-1-yl-benzidine (α-NPD).

Due to their good charge transfer characteristics, the use of triarylamine derivatives, particularly triarylamine dimers, in electrophotographic and electroluminescent applications has been known for a long time. Especially N,N′-bis(-4′-N,N-diphenylamino-biphenylyl))-N,N′-diphenyl-benzidine (EP0650955A1) and N,N′-bis(-4′(-N-phenyl-N-naphth-1 yl-amino-biphenylyl))-N,N′-diphenyl-benzidine (JP2000260572) are used, either alone or combined with TPD or α-NPD in a double layer structure.

In general, the service life and the efficiency, or its development as time passes, of the known electroluminescent devices do not meet the requirements of practice and need to be improved. The film forming characteristics of the charge transfer materials used and their morphological stability within a binder layer are also unsatisfactory. In particular, the tendency of a layer containing the aforesaid charge transfer materials to form crystallization centres within the said layer during the service life of an electroluminescent device or arrangement largely depends on the glass transition temperature of the materials used. The higher the glass transition temperature the lower is, in general, the recrystallization tendency at a given temperature, while at the same time the speed of crystallization below the glass transition temperature is extremely low. Therefore, arrangements manufactured using compounds whose glass transition temperature is high can be expected to have a high permissible working temperature.

A high glass transition temperature is highly favoured by the existence of space-filling, sterically demanding groups.

The present application provides new compounds which are suitable as charge transfer materials and whose glass transition temperatures are in the range from 100° C., preferably 150° C., to 250° C. thus extending the operative range of the electroluminescent arrangements manufactured using the aforesaid compounds to temperatures ranging from 100° C. to approx. 200° C.

According to one embodiment, the new triarylamine derivatives correspond to the general formula 1 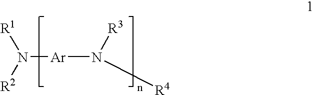
where
n is an integer from 1 to 10;
R¹, R², R³ and R⁴, which are the same or different, are phenyl, biphenylyl, methylphenyl, naphthyl, phenanthrenyl, anthracenyl, fluorenyl, tri-arylmethyl-aryl or triarylsilyl-aryl,
at least one of the rests R¹ through R⁴ being triarylmethyl-aryl or triarylsilyl-aryl according to formula 4

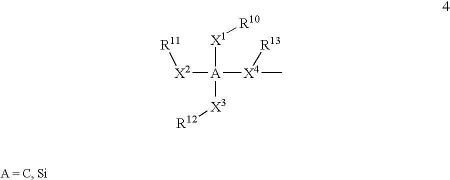

where the aromatic or heteroaromatic units X¹ through X⁴, which are the same or different, are phenyl, naphthyl, anthracenyl, phenanthrenyl, pyrenyl, pyridyl or chinolyl, and where R¹⁰, R¹¹, R¹² and R¹³, which are the same or different, have the meaning of H, C₁, to C₆ alkyl, cycloalkyl, C₂ to C₄ alkenyl, C₁, to C₄ alkoxy, C₁ to C₄ di-alkylamino, diarylamino, halogen, hydroxy, phenyl, naphthyl or pyridyl,
and where R¹ through R⁴ having the meaning of phenyl, biphenylyl, methylphenyl, naphthyl, phenanthrenyl, anthracenyl, fluorenyl can be substituted by one or more of the substituents C₁ to C₃ alkyl, C₁ to C₂ alkoxy or halogen;
Ar is a structure according to formula 2 or 3 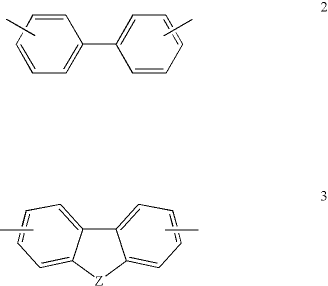
which structure Ar can be the same or different if n>1 and
where Z in formula 3 is selected from among the following structures 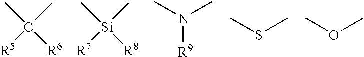
where R⁵ through R⁹, which are the same or different, are H or C₁ to C₁₅ alkyl, or R₅ and R₆ or R₇ and R₈ combine to form a 5-membered or 6-membered alicyclic or heterocyclic ring thus forming a spiro ring system together with the five-membered ring they are bonded to, wherein O, N or S can be the heterocyclic elements;
or Ar is a structure according to formula 29, 30, 31 or 32 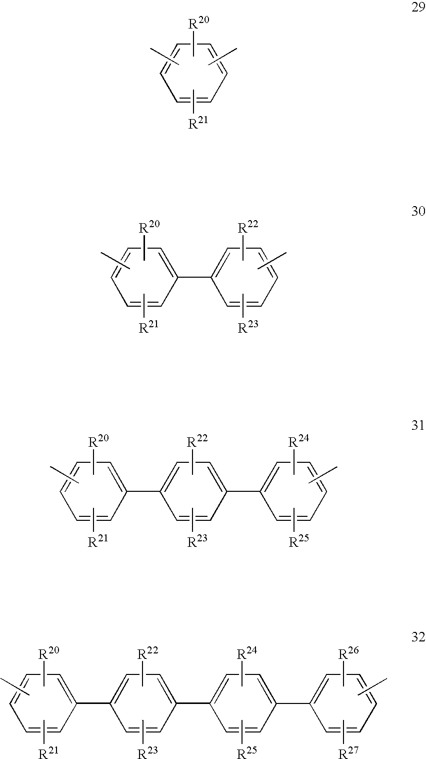
and where R²⁰ through R²⁷, which are the same or different, have the meaning of H, phenyl, C₁ to C₅ alkyl or C₁ to C₃ alkoxy and the structures 29, 30, 31 or 32 are connected to the adjacent nitrogen atoms in any free substitution position, on condition that, if n=1 or 2 and Ar is biphenylene or one of the groups according to formulas 29 through 32, at least one of the rests R¹ through R⁴ be a triarylsilyl-aryl rest or a substituted triarylmethyl-aryl unit according to the above formula 4, R¹⁰ through R¹² having the meaning specified above.

Preferred triarylamine derivatives are those according to formula 1, where n is a whole number between 1 and 4, particularly 1 or 2.

Preferred rests R¹ through R⁴ in formula 1 have the meaning of phenyl, bi-phenylyl, methylphenyl, naphthyl, fluorenyl, triarylmethyl-aryl or triarylsilyl-aryl.

Preferred rests R⁵ through R⁹, which can be the same or different, have the meaning of methyl or phenyl.

In another embodiment of the invention, the rests R⁵ and R⁶ form a spiro alkane ring together with the C atom they are bonded to.

Preferred rests R²⁰ through R²⁷, which can be the same or different, are hydrogen, methyl or phenyl.

If the structures Ar include at least one unit according to formula 3, it is preferred that at least one of the rests R¹ through R⁴ be a triarylsilyl-aryl unit or a substituted triarylmethyl-aryl unit according to formula 4.

If all the structures Ar consist of units according to formula 2, it is preferred that at least one of the rests R¹ through R⁴ be
a triarylsilyl-aryl unit according to formula 4, or a triarylmethyl-aryl unit according to formula 4, on condition that in this case at least one of the rests R¹⁰ through R¹³ is not H, or a triarylmethyl-aryl unit according to formula 4, on condition that in this case at least one of the rests X¹ through X⁴ be a heteroaromatic substance.

The rests R¹⁰ through R¹³ are preferably H, phenyl, C₁ to C₃ alkyl, C₁ to C₃ alkoxy or halogen. Methyl or phenyl are particularly preferred. The halogen is preferably F or Cl.

A preferred embodiment of the invention relates to triarylamine derivatives according to the general formula 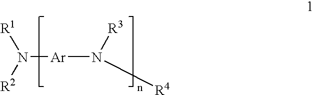
where
n is an integer from 1 to 10;
R¹, R², R³ and R⁴, which are the same or different, are phenyl, biphenyl, methylphenyl, naphthyl, phenanthrenyl, anthracenyl, fluorenyl, triphenylmethyl or triphenylsilyl, wherein at least one of the rests R¹ through R⁴is a phenyl group substituted with a triphenylmethyl or triphenylsilyl group according to formula 33 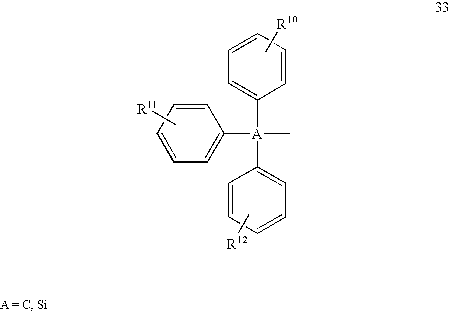
where R¹⁰, R¹¹ and R¹², which are the same or different, have the meaning of H, C₁ to C₆ alkyl, cycloalkyl, C₂ to C₄ alkenyl, C₁ to C₄ alkoxy or halogen, and where R¹ through R⁴ can be substituted by one or more substituent(s);
Ar is 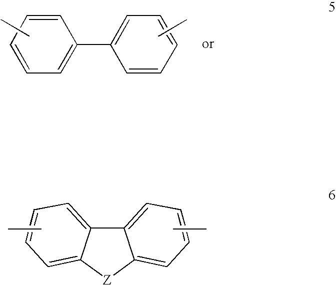
where Z is selected from among the following structures 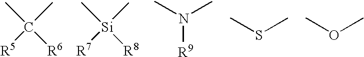
where R⁵ through R⁹, which are the same or different, are H or C₁ to C₅ alkyl, on condition that, if n=1 and Ar is biphenyl according to formula 5, at least one of the rests R¹ through R⁴ be a triphenylsilyl rest according to the above formula 4, R¹⁰ through R¹² having the meaning specified above. The preferred meanings of Ar and R¹ through R²⁷ specified above apply to this embodiment too.

The application further relates to an organic electroluminescent device having at least one hole transport layer and one luminescent layer, wherein at least one hole transport layer contains a triarylamine derivative according to formula 1.

Another embodiment consists in that the organic electroluminescent device comprises a luminescent layer containing a triarylamine derivative according to formula 1.

The application also relates to the use of triarylamine derivatives according to formula 1 as hole transfer substance or luminescent substance in an organic electroluminescent device, and the use of triarylamine derivatives according to formula 1 as hole transfer substance in an electrophotographic arrangement.

Typically, an electrophotographic device has the following structure. A charge generation layer is arranged above an electrically conductive metal layer, which can either be applied onto a flexible substrate or consist of an aluminium drum, which charge generation layer has the task of injecting positive charge carriers into the charge transfer layer during exposure. The arrangement is charged electrostatically up to several hundred volts before exposure. The charge generation layer and the charge transfer layer are typically between 15 and 25 μm thick, and under the influence of the high field strength caused by the aforesaid process, the positive charge carriers (electron “holes”) injected move towards the negatively charged charge transfer layer thus bringing about a discharge of the surface in those areas onto which light has fallen. In the subsequent steps of an electrophotographic cycle, toner is applied onto the surface which is charged (or discharged) according to the picture, the toner is transferred onto a printing material, if necessary, fixed on the aforesaid material, and finally excess toner and the residual charge are removed.

An electroluminescent device, in principle, consists of one or more charge transfer layer(s) which contain(s) an organic compound and is/are arranged between two electrodes of which at least one is transparent. If a voltage is applied, the metal electrode (mostly Ca, Mg or Al, often in combination with silver), whose work function is low, injects electrons, and the opposite electrode injects holes into the organic layer, which electrons and holes combine to form singlet excitons. The latter return to their normal state after a short while thereby emitting light.

An additional separation of the electron transfer layer and the electroluminescent layer brings about an increase in quantum efficiency. At the same time, the electroluminescent layer can be selected to be very thin. As the flourescent material can be replaced regardless of its electron transfer behaviour, the emission wavelength can be set in the whole visible spectral range in a targeted manner.

It is also possible to split the hole transport layer into two partial layers which differ in composition.

According to one embodiemnt, the organic electroluminescent device consists of a combination of layers consisting of a cathode, an electroluminescent layer containing an organic compound, and an anode, the organic compound contained in the hole transport layer being a triarylamine derivative according to the general formula 1.

A preferred structure consists of the following layers:
substrate, transparent anode, hole transport layer, electroluminescent layer, electron transfer layer, cathode. The cathode, which can consist of Al, Mg, In, Ag or alloys of the aforesaid metals, has a thickness ranging from 100 to 5000 Å. The transparent anode can consist of indium tin oxide (ITO) having a thickness of between 1000 and 3000 Å, an indium antimony tin oxide coating or a semitransparent gold layer applied onto a glass substrate.

The electroluminescent layer, which contains tris(-8-hydroxychinolino)-aluminium according to the formula 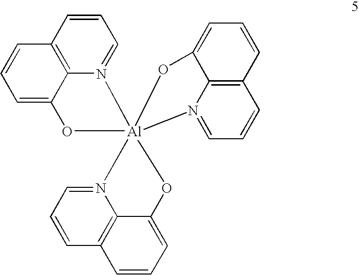
as usual luminescent substance, in some cases contains further fluorescent substances, e.g. substituted triphenylbutadienes and/or 1,3,4-oxadiazol derivatives, distyrylarylene derivatives, chinacridones, salicylidene Zn complexes, zinc chelate complexes, aluminium chelate complexes doped with DCM, squarine derivatives, 9,10-bis-styrylanthracene derivatives or europium complexes. However, the electroluminescent layer may only also contain luminescent compounds of the type described herein and/or may contain mixtures thereof with known luminescent substances.

Typical examples of triarylamine derivatives according to the general formula 1 are:

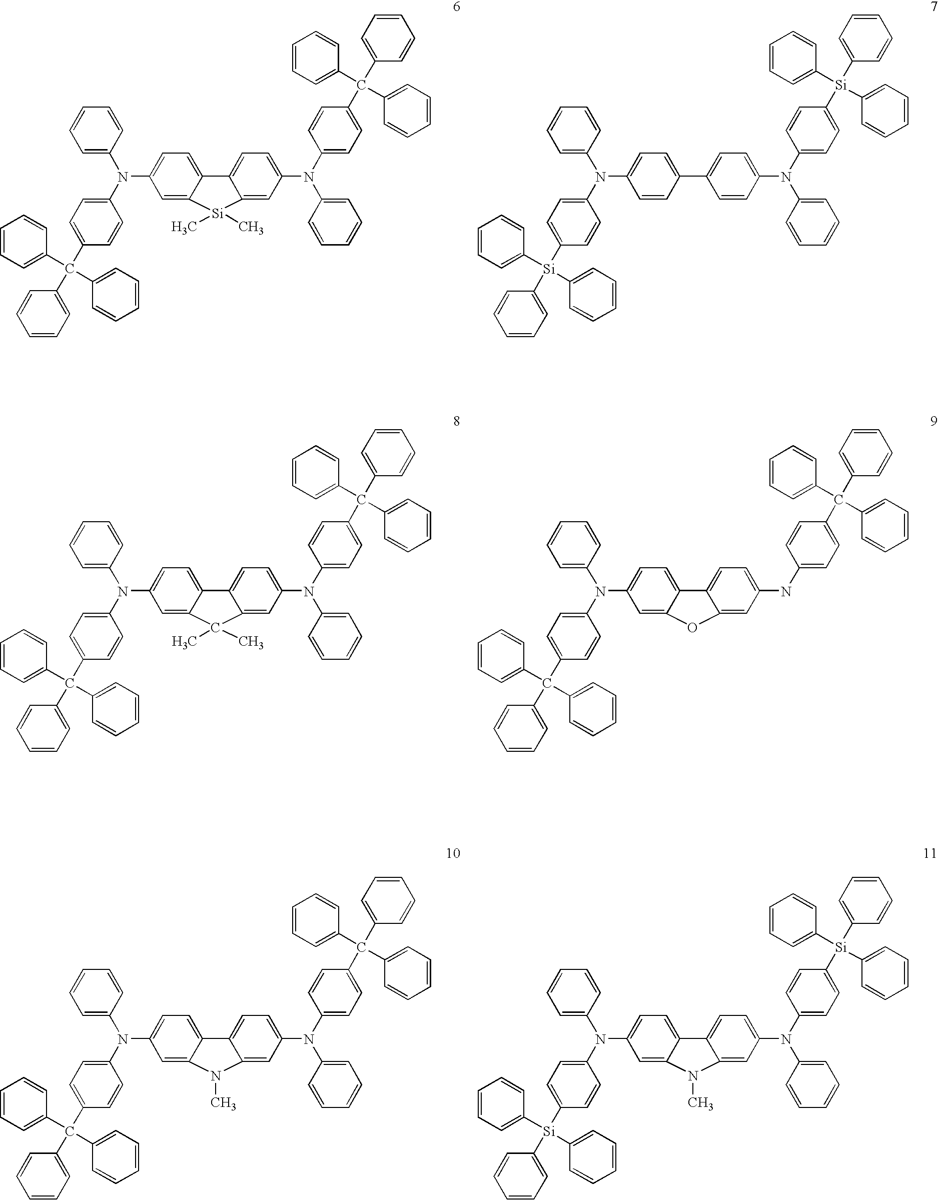

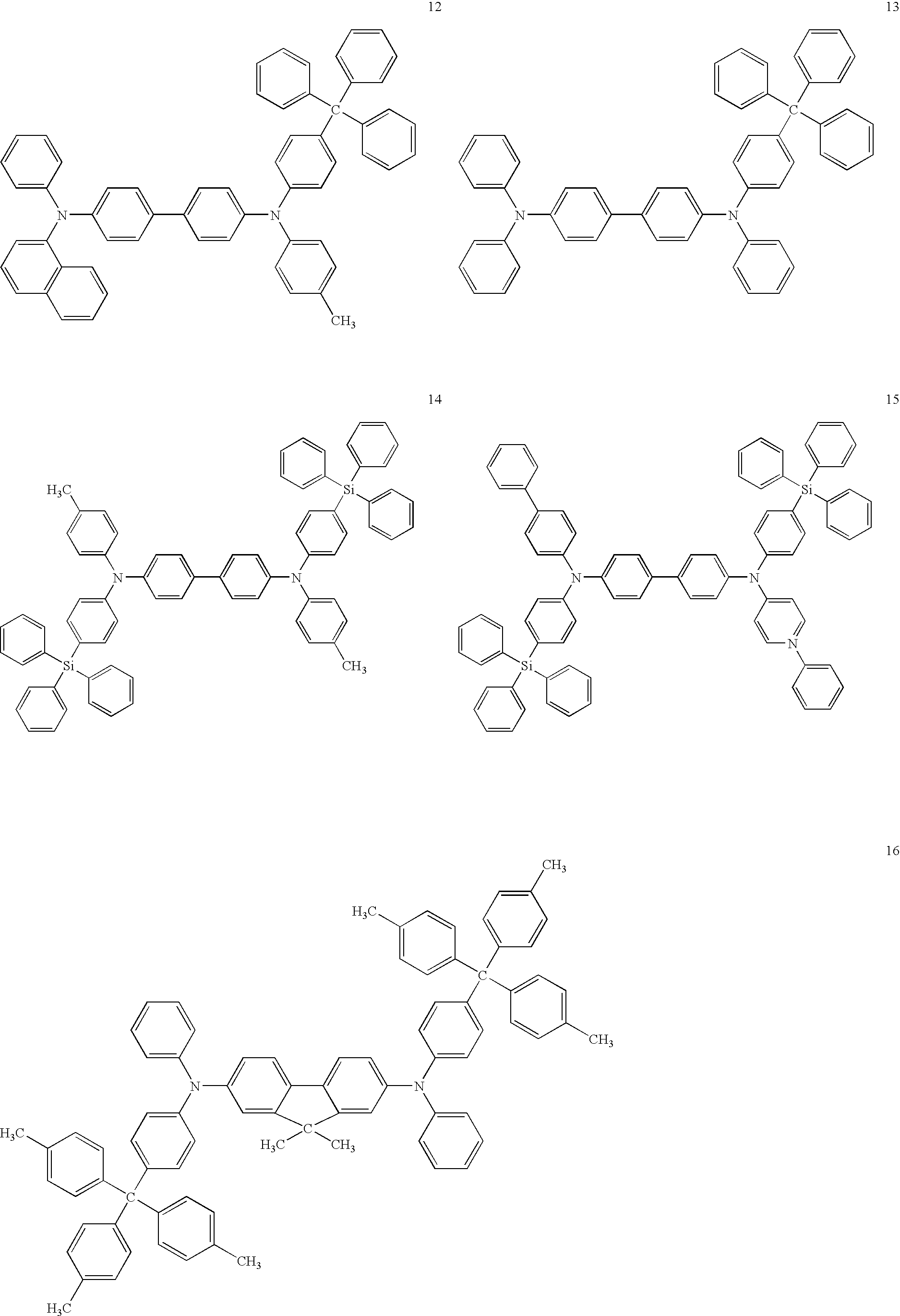

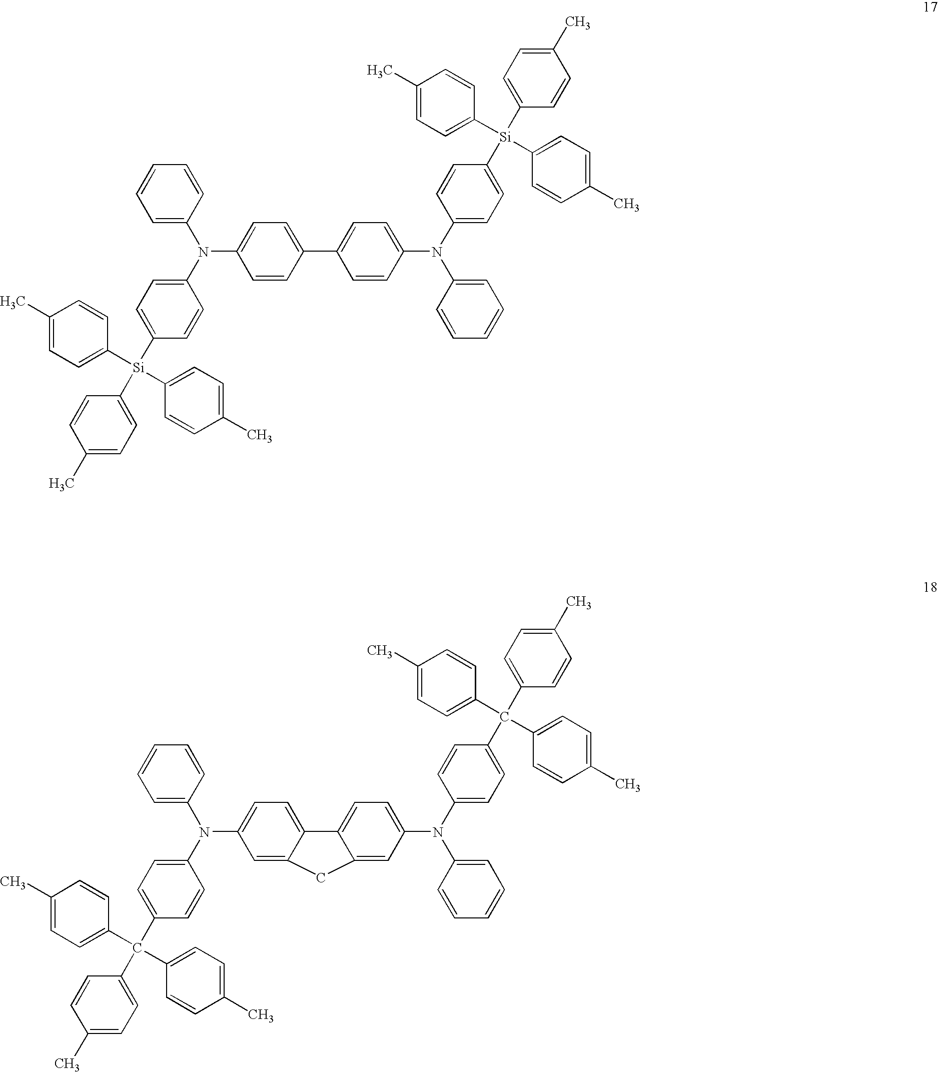

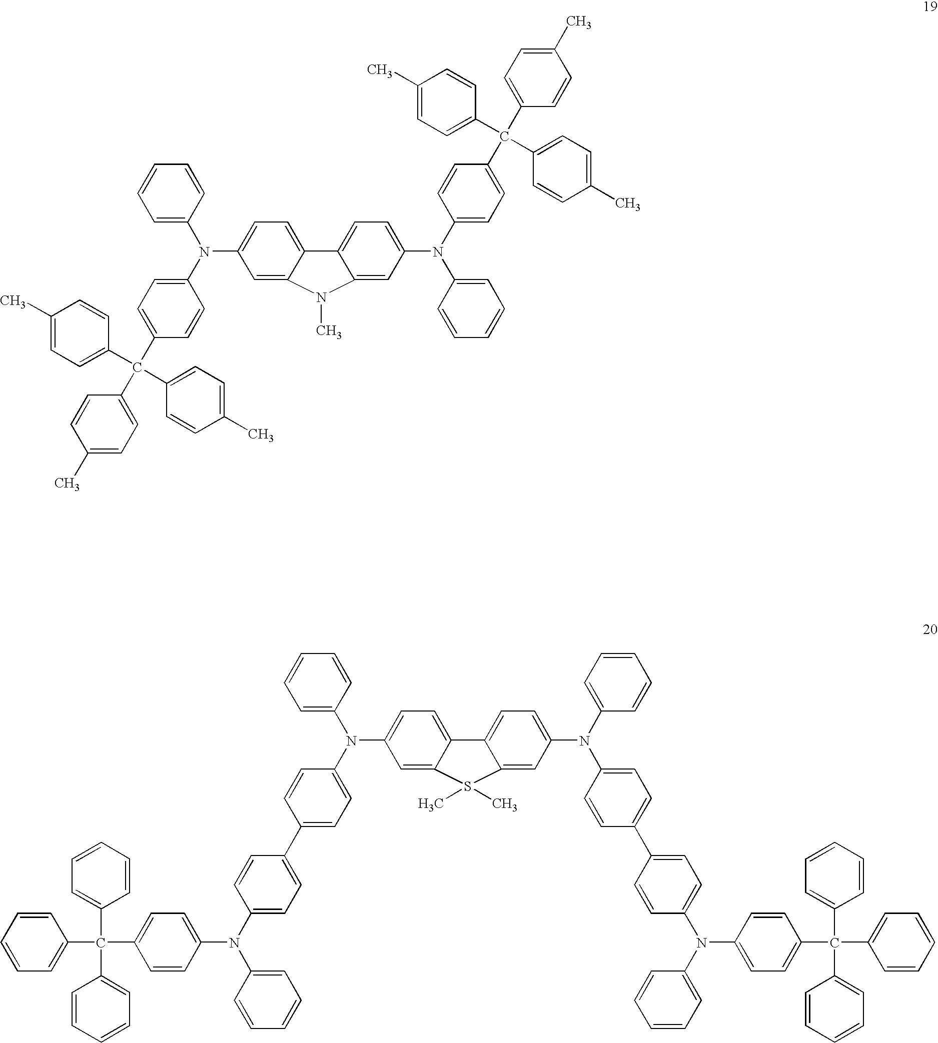

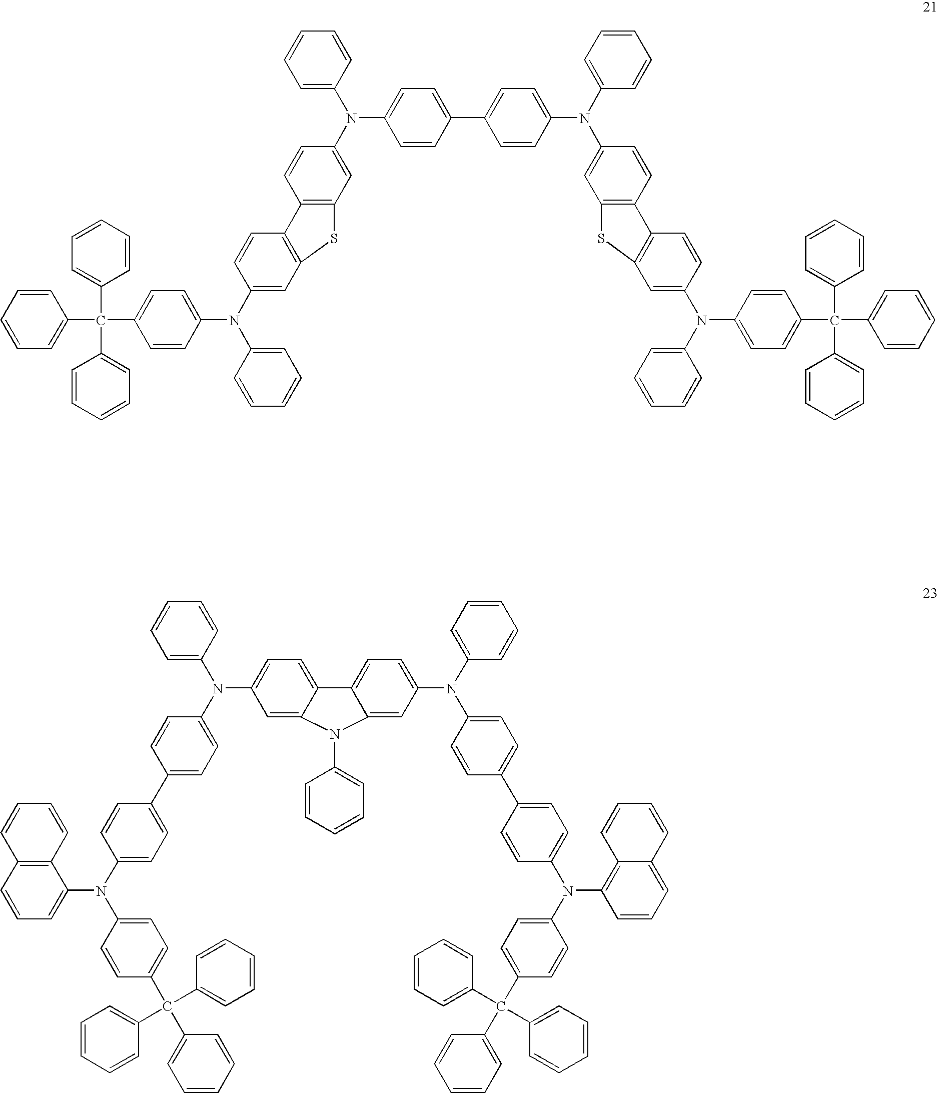

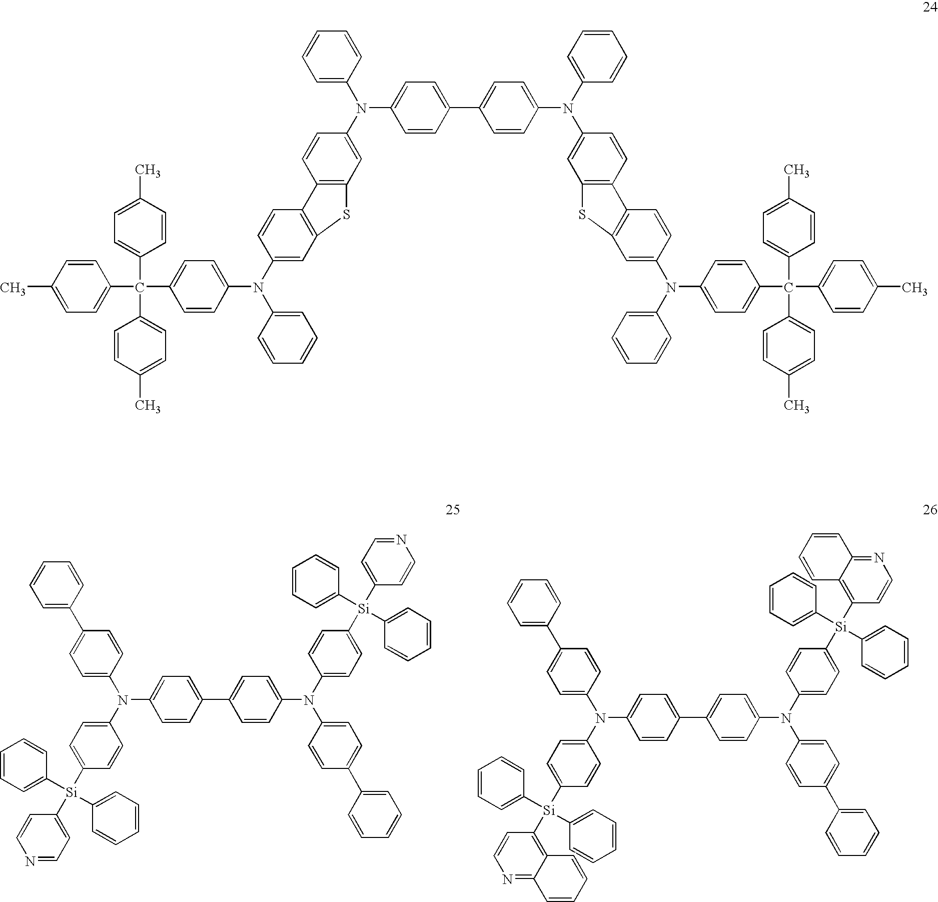

The following tables 1 and 2 indicate preferred embodiments for the structural units Ar and the rests R^{x} (R¹ through R⁴) according to formula 1.

TABLE 1
Ar

| | |
|---|---|
| 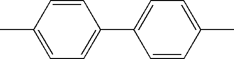 | 001 |
| 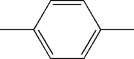 | 002 |
| 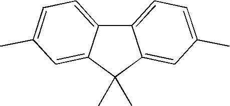 | 003 |
| 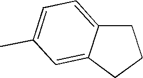 | 004 |
| 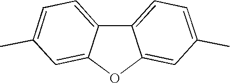 | 005 |
| 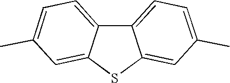 | 006 |
| 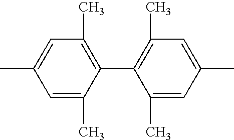 | 007 |
| 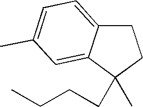 | 008 |
| 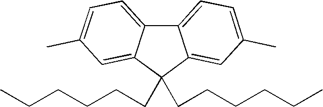 | 009 |
| 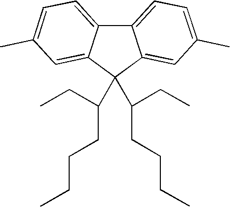 | 010 |
| 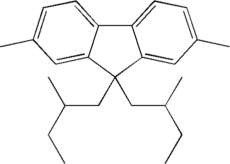 | 011 |
| 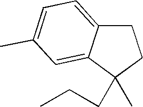 | 012 |
| 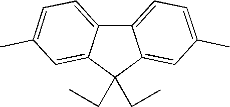 | 013 |
| 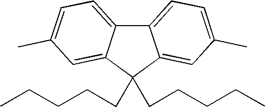 | 014 |

TABLE 2
R^{X}

| | |
|---|---|
| 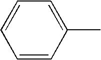 | 100 |
| 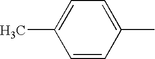 | 101 |
| 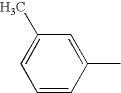 | 102 |
| 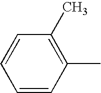 | 103 |
| 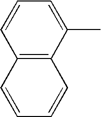 | 104 |
| 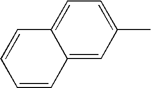 | 105 |
| 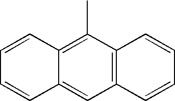 | 106 |
| 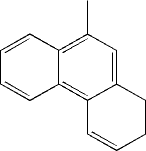 | 107 |
| 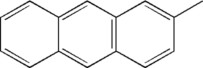 | 108 |
| 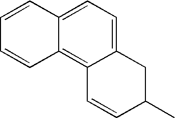 | 109 |
| 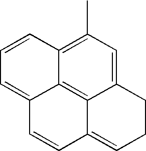 | 110 |
| 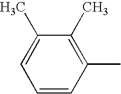 | 111 |
| 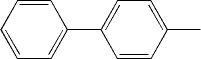 | 112 |
| 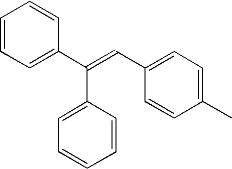 | 113 |
| 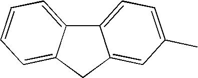 | 114 |
| 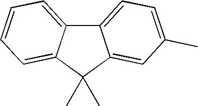 | 115 |
| 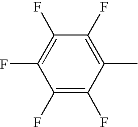 | 116 |
| 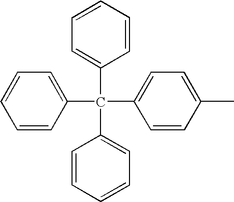 | 117 |
| 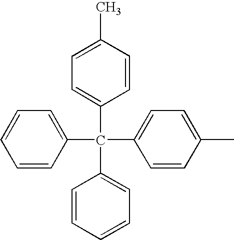 | 118 |
| 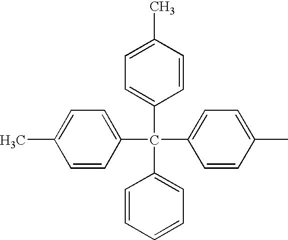 | 119 |
| 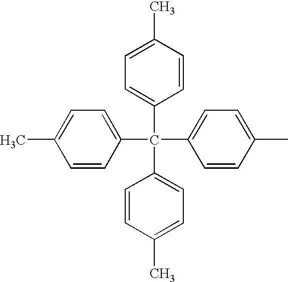 | 120 |
| 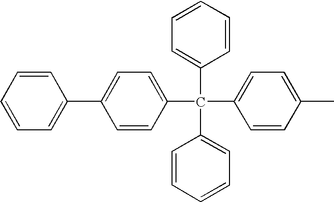 | 121 |
| 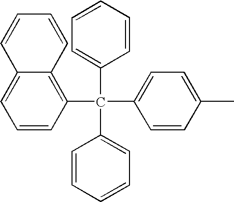 | 122 |
| 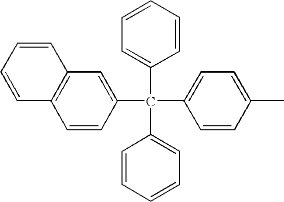 | 123 |
| 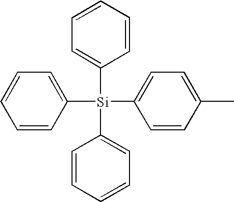 | 124 |
| 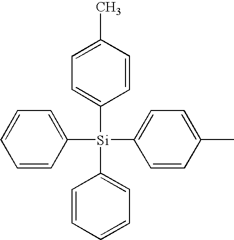 | 125 |
| 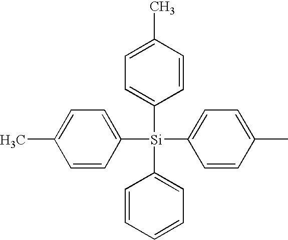 | 126 |
| 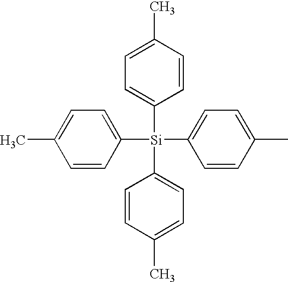 | 127 |
| 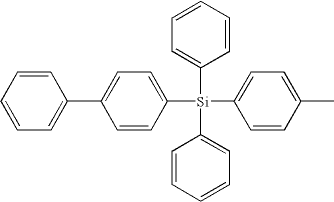 | 128 |
| 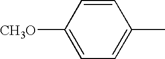 | 129 |
| 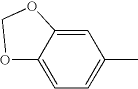 | 130 |

Based on the above tables for Ar and R^{x}, the following tables 3, 4 and 5 indicate the composition of preferred specific example compounds according to the general formula 1 for different n values.

TABLE 3
n = 1:

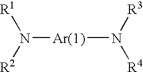

| R¹ | R² | Ar(1) | R³ | R⁴ |
|---|---|---|---|---|
| 100 | 118 | 001 | 100 | 118 |
| 100 | 119 | | 100 | 119 |
| 100 | 120 | | 100 | 120 |
| 100 | 121 | | 100 | 121 |
| 100 | 122 | | 100 | 122 |
| 100 | 123 | | 100 | 123 |
| 100 | 124 | | 100 | 124 |
| 100 | 125 | | 100 | 125 |
| 100 | 126 | | 100 | 126 |
| 100 | 127 | | 100 | 127 |
| 100 | 128 | | 100 | 128 |
| 101 | 120 | | 100 | 100 |
| 101 | 121 | | 101 | 121 |
| 101 | 122 | | 101 | 122 |
| 101 | 123 | | 101 | 123 |
| 101 | 124 | | 101 | 124 |
| 101 | 125 | | 101 | 125 |
| 101 | 126 | | 101 | 126 |
| 101 | 127 | | 101 | 127 |
| 101 | 128 | | 101 | 128 |
| 102 | 123 | | 102 | 123 |
| 102 | 124 | | 102 | 124 |
| 103 | 120 | | 103 | 120 |
| 105 | 120 | | 105 | 120 |
| 107 | 121 | | 107 | 121 |
| 110 | 119 | | 110 | 119 |
| 111 | 124 | | 111 | 124 |
| 111 | 128 | | 111 | 128 |
| 112 | 118 | | 112 | 118 |
| 112 | 119 | | 112 | 119 |
| 112 | 120 | | 112 | 120 |
| 112 | 121 | | 112 | 121 |
| 112 | 122 | | 112 | 122 |
| 112 | 123 | | 112 | 123 |
| 112 | 124 | | 112 | 124 |
| 112 | 125 | | 112 | 125 |
| 112 | 126 | | 112 | 126 |
| 112 | 127 | | 112 | 127 |
| 112 | 128 | | 112 | 128 |
| 113 | 124 | | 113 | 124 |
| 115 | 124 | | 115 | 124 |
| 124 | 124 | | 124 | 124 |
| 129 | 127 | | 129 | 127 |
| 129 | 128 | | 129 | 128 |
| 100 | 120 | 002 | 100 | 120 |
| 100 | 124 | | 100 | 124 |
| 100 | 128 | | 100 | 128 |
| 102 | 124 | | 102 | 124 |
| 100 | 117 | 003 | 100 | 117 |
| 100 | 124 | | 100 | 124 |
| 100 | 118 | | 100 | 118 |
| 101 | 117 | | 102 | 117 |
| 101 | 124 | | 101 | 124 |
| 103 | 120 | | 103 | 120 |
| 103 | 124 | | 103 | 124 |
| 112 | 121 | | 112 | 121 |
| 100 | 117 | 004 | 100 | 117 |
| 100 | 124 | | 100 | 124 |
| 101 | 117 | | 101 | 117 |
| 100 | 117 | 005 | 100 | 117 |
| 100 | 124 | | 100 | 124 |
| 101 | 123 | | 101 | 123 |
| 101 | 117 | | 101 | 117 |
| 103 | 117 | | 103 | 117 |
| 103 | 122 | | 103 | 122 |
| 100 | 117 | 006 | 100 | 117 |
| 100 | 124 | | 100 | 124 |
| 101 | 123 | | 101 | 123 |
| 101 | 117 | | 101 | 117 |
| 103 | 117 | | 103 | 117 |
| 103 | 122 | | 103 | 122 |
| 100 | 117 | 007 | 100 | 117 |
| 100 | 121 | | 100 | 121 |
| 101 | 123 | | 101 | 123 |
| 103 | 118 | | 103 | 118 |
| 100 | 117 | 008 | 100 | 117 |
| 100 | 124 | | 100 | 124 |
| 101 | 124 | | 101 | 124 |
| 100 | 117 | 009 | 100 | 117 |
| 100 | 124 | | 100 | 124 |
| 101 | 124 | | 101 | 124 |
| 100 | 124 | 009 | 100 | 124 |
| 100 | 117 | | 100 | 117 |
| 102 | 117 | | 102 | 117 |
| 102 | 124 | | 102 | 124 |
| 100 | 117 | 010 | 100 | 117 |
| 100 | 124 | | 100 | 124 |
| 101 | 124 | | 101 | 124 |
| 100 | 117 | 011 | 100 | 117 |
| 100 | 124 | | 100 | 124 |
| 101 | 124 | | 101 | 124 |
| 100 | 117 | 012 | 100 | 117 |
| 100 | 124 | | 100 | 124 |
| 101 | 124 | | 101 | 124 |
| 100 | 117 | 013 | 100 | 117 |
| 100 | 124 | | 100 | 124 |
| 101 | 124 | | 101 | 124 |
| 100 | 117 | 014 | 100 | 117 |
| 100 | 124 | | 100 | 124 |
| 101 | 124 | | 101 | 124 |

TABLE 4
n = 2:

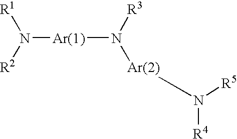

| R¹ | R² | Ar(1) | R₃ | Ar(2) | R⁴ | R⁵ |
|---|---|---|---|---|---|---|
| 100 | 124 | 001 | 100 | 001 | 124 | 100 |
| 101 | 120 | | 101 | | 120 | 101 |
| 101 | 120 | | 120 | | 120 | 101 |
| 104 | 124 | | 100 | | 124 | 104 |
| 105 | 123 | | 100 | | 123 | 105 |
| 106 | 124 | | 100 | | 124 | 106 |
| 107 | 120 | | 101 | | 120 | 107 |
| 112 | 118 | | 100 | | 118 | 112 |
| 100 | 124 | 001 | 100 | 003 | 124 | 100 |
| 100 | 117 | | 100 | | 117 | 100 |
| 101 | 120 | | 101 | | 120 | 101 |
| 101 | 120 | | 120 | | 120 | 101 |
| 101 | 117 | | 101 | | 117 | 101 |
| 102 | 117 | | 100 | | 117 | 102 |
| 103 | 117 | | 100 | | 117 | 103 |
| 104 | 124 | | 100 | | 124 | 104 |
| 105 | 123 | | 100 | | 123 | 105 |
| 106 | 124 | | 100 | | 124 | 106 |
| 107 | 120 | | 101 | | 120 | 107 |
| 112 | 118 | | 100 | | 118 | 112 |
| 100 | 124 | 001 | 100 | 005 | 124 | 100 |
| 100 | 117 | | 100 | | 117 | 100 |
| 101 | 120 | | 101 | | 120 | 101 |
| 101 | 120 | | 120 | | 120 | 101 |
| 101 | 117 | | 101 | | 117 | 101 |
| 102 | 117 | | 100 | | 117 | 102 |
| 103 | 117 | | 100 | | 117 | 103 |
| 104 | 124 | | 100 | | 124 | 104 |
| 105 | 123 | | 100 | | 123 | 105 |
| 106 | 124 | | 100 | | 124 | 106 |
| 107 | 120 | | 101 | | 120 | 107 |
| 112 | 118 | | 100 | | 118 | 112 |
| 100 | 124 | 001 | 100 | 006 | 124 | 100 |
| 100 | 117 | | 100 | | 117 | 100 |
| 101 | 120 | | 101 | | 120 | 101 |
| 101 | 120 | | 120 | | 120 | 101 |
| 101 | 117 | | 101 | | 117 | 101 |
| 102 | 117 | | 100 | | 117 | 102 |
| 103 | 117 | | 100 | | 117 | 103 |
| 104 | 124 | | 100 | | 124 | 104 |
| 105 | 123 | | 100 | | 123 | 105 |
| 106 | 124 | | 100 | | 124 | 106 |
| 107 | 120 | | 101 | | 120 | 107 |
| 112 | 118 | | 100 | | 118 | 112 |
| 100 | 117 | 003 | 100 | 003 | 117 | 100 |
| 101 | 124 | | 100 | | 124 | 101 |
| 104 | 124 | | 100 | | 124 | 104 |
| 100 | 121 | 007 | 100 | 007 | 121 | 100 |
| 100 | 124 | | 100 | | 124 | 100 |
| 103 | 118 | | 100 | | 118 | 103 |
| 101 | 121 | 001 | 100 | 007 | 121 | 101 |

TABLE 5
n = 3:

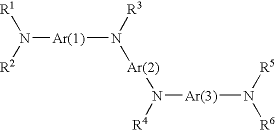

| R¹ | R² | Ar(1) | R³ | Ar(2) | R⁴ | Ar(3) | R⁵ | R⁶ |
|---|---|---|---|---|---|---|---|---|
| 100 | 124 | 001 | 100 | 001 | 100 | 001 | 124 | 100 |
| 104 | 124 | | 100 | | 100 | | 124 | 104 |
| 105 | 124 | | 100 | | 100 | | 124 | 105 |
| 100 | 117 | 001 | 100 | 003 | 100 | 001 | 117 | 100 |
| 101 | 120 | | 100 | | 100 | | 120 | 101 |
| 104 | 120 | | 100 | | 100 | | 120 | 104 |
| 104 | 124 | | 100 | | 100 | | 124 | 104 |
| 104 | 124 | | 101 | | 101 | | 124 | 104 |
| 108 | 120 | | 100 | | 100 | | 120 | 108 |
| 110 | 120 | | 100 | | 100 | | 120 | 110 |
| 100 | 117 | 003 | 100 | 001 | 100 | 003 | 117 | 100 |
| 101 | 120 | | 100 | | 100 | | 120 | 101 |
| 104 | 120 | | 100 | | 100 | | 120 | 104 |
| 104 | 124 | | 100 | | 100 | | 124 | 104 |
| 104 | 124 | | 101 | | 101 | | 124 | 104 |
| 108 | 120 | | 100 | | 100 | | 120 | 108 |
| 110 | 120 | | 100 | | 100 | | 120 | 110 |
| 100 | 117 | 001 | 100 | 005 | 100 | 001 | 117 | 100 |
| 101 | 120 | | 100 | | 100 | | 120 | 101 |
| 104 | 120 | | 100 | | 100 | | 120 | 104 |
| 104 | 124 | | 100 | | 100 | | 124 | 104 |
| 104 | 124 | | 101 | | 101 | | 124 | 104 |
| 108 | 120 | | 100 | | 100 | | 120 | 108 |
| 110 | 120 | | 100 | | 100 | | 120 | 110 |
| 100 | 117 | 005 | 100 | 001 | 100 | 005 | 117 | 100 |
| 101 | 120 | | 100 | | 100 | | 120 | 101 |
| 104 | 120 | | 100 | | 100 | | 120 | 104 |
| 104 | 124 | | 100 | | 100 | | 124 | 104 |
| 104 | 124 | | 101 | | 101 | | 124 | 104 |
| 108 | 120 | | 100 | | 100 | | 120 | 108 |
| 110 | 120 | | 100 | | 100 | | 120 | 110 |
| 100 | 117 | 005 | 100 | 006 | 100 | 005 | 117 | 100 |
| 100 | 124 | | 100 | | 100 | | 124 | 100 |
| 104 | 117 | | 100 | | 100 | | 117 | 104 |
| 112 | 117 | | 100 | | 100 | | 117 | 112 |
| 100 | 117 | 006 | 100 | 005 | 100 | 006 | 117 | 100 |
| 100 | 124 | | 100 | | 100 | | 124 | 100 |
| 104 | 117 | | 100 | | 100 | | 117 | 104 |
| 112 | 117 | | 100 | | 100 | | 117 | 112 |
| 100 | 117 | 001 | 100 | 013 | 100 | 001 | 117 | 100 |
| 101 | 120 | | 100 | | 100 | | 120 | 101 |
| 104 | 120 | | 100 | | 100 | | 120 | 104 |
| 104 | 124 | | 100 | | 100 | | 124 | 104 |
| 104 | 124 | | 101 | | 101 | | 124 | 104 |
| 108 | 120 | | 100 | | 100 | | 120 | 108 |
| 110 | 120 | | 100 | | 100 | | 120 | 110 |
| 100 | 117 | 001 | 100 | 014 | 100 | 001 | 117 | 100 |
| 101 | 120 | | 100 | | 100 | | 120 | 101 |
| 104 | 120 | | 100 | | 100 | | 120 | 104 |
| 104 | 124 | | 100 | | 100 | | 124 | 104 |
| 104 | 124 | | 101 | | 101 | | 124 | 104 |
| 108 | 120 | | 100 | | 100 | | 120 | 108 |
| 110 | 120 | | 100 | | 100 | | 120 | 110 |
| 100 | 117 | 001 | 100 | 007 | 100 | 001 | 117 | 100 |
| 101 | 120 | | 100 | | 100 | | 120 | 101 |
| 104 | 120 | | 100 | | 100 | | 120 | 104 |
| 104 | 124 | | 100 | | 100 | | 124 | 104 |
| 104 | 124 | | 101 | | 101 | | 124 | 104 |
| 108 | 120 | | 100 | | 100 | | 120 | 108 |
| 110 | 120 | | 100 | | 100 | | 120 | 110 |
| 100 | 117 | 007 | 100 | 001 | 100 | 007 | 117 | 100 |
| 101 | 120 | | 100 | | 100 | | 120 | 101 |
| 104 | 120 | | 100 | | 100 | | 120 | 104 |
| 104 | 124 | | 100 | | 100 | | 124 | 104 |
| 104 | 124 | | 101 | | 101 | | 124 | 104 |
| 108 | 120 | | 100 | | 100 | | 120 | 108 |
| 110 | 120 | | 100 | | 100 | | 120 | 110 |

The new compounds are synthesized according to methods known per se, e.g. according to the Ullmann Synthesis or by means of reaction processes which use noble metal catalysts and are based on suitable primary and secondary amines and (according to formulas 2 and 3) dihalogen-biphenyls, dihalogen-dibenzofurans, dihalogen-dibenzothiophenes, dihalogencarbazols or dihalogen-dibenzosilols, or on suitable tertiary halogen-biphenyl-4-yl-amines and (according to formulas 2 or 3) heteroanalogous benzidine derivatives.

The Ullmann Synthesis is a condensation reaction in which aryl halogenides, preferably aryl iodides, react with suitable substrates to form C arylation products or N arylation products at temperatures ranging from 100° C. to 300° C. and using Cu or Cu bronze as catalyst, wherein functionally substituted aryl halogenides can also be reacted if sensitive groups are selectively protected.

If two hole transport layers arranged one after the other are used, at least one layer contains triarylamine derivatives according to formula 1, preferably one or more compound(s) 6-24.

If an additional electron transfer layer is used, it contains known electron transfer materials, e.g. bis(-aminophenyl)-1,3,4-oxadiazols, triazols or dithiolene derivatives.

The use of hole transfer materials according to formulas 6 through 24 brings about a high dark conductivity of the layers and thus a low turn-on voltage of less than 6 volts, which results in a reduction of the thermal stress exerted on the device. At the same time, the hole transfer materials used in embodiments of the present devices have a high glass transition temperature of more than 150° C. up to 250° C. and thus a very low tendency to recrystallize in the layer. Due to the aforesaid characteristics and due to the chemical structure of these relatively large molecules, layers produced of these substances are very stable, no matter whether they contain binder or not, which enables the common spin-coating technique to be used.

Layers applied by means of vacuum metallization are free from structural defective spots and have a high transparency in the visual spectral range. The aforesaid characteristics enable new organic electroluminescent devices to be produced, which have a high luminance (>10,000 cd/m²) and, at the same time, a considerably improved long-term stability (>10,000 hours). The working range of the aforesaid devices is in the temperature range from 100 to 200° C., preferably 120 to 200° C., particularly 120 to 150° C.

The following examples are intended to illustrate the present invention without limiting it in any way:

### EXAMPLE 1

Production of N,N′-bis-(4′-(N-triphenylmethyl)-phenyl)-N-naphth-1-yl-amino)-bi-phenylyl)-N,N′-bisphenyl-2,7-amino-9-phenylcarbazol (formula 23)

A glass apparatus consisting of a 500 ml three-necked flask which is provided with a reflux condenser, a magnetic stirrer, a thermometer and a gas inlet pipe is heated at a temperature of 120° C. for 2 hours in order to remove the water adherent to the glass walls.

In a nitrogen atmosphere, 160 ml o-xylol which has been dried over Na and swept with N₂ is supplied into the apparatus. 6.3 mg palladium acetate and 5.2 ml of a 1% solution of tri-tert.-butylphosphine in dry o-xylol are added while stirring, which results in the catalyst complex being formed.

12.9 g sodium-tert.-butylate, 23.8 g 2,7-dianilino-N-phenylcarbazol and 69.1 g N-triphenylmethyl-phenyl-N-naphth-1-yl-(4-bromobiphenylyl)-amine are added into the clear yellow solution produced.

The nitrogen atmosphere is maintained and the flask's content is heated up to 120° C. in an oil bath while stirring. NaBr starts to precipitate after approx. 30 min. The mixture is left to react for 3 hours at a temperature of 120° C. Subsequently, the flask's content is diluted to twice its volume by adding toluol, and then added into the tenfold amount of methanol while stirring. During the aforesaid step, the raw product is precipitated and can be separated by means of filtration.

In order to clean the raw product, it is re-precipitated out of dodecane and subsequently recrystallized out of DMF. Finally, the product is sublimed under an ultra-high vacuum (<10−⁵ torrs). In this way, approx. 30 g pure N,N′-bis-(4′-(N-tri-phenylmethyl)-phenyl)-N-naphth-1-yl-amino)-biphenylyl)-N,N′-bisphenyl-2,7-amino-N-phenylcarbazol is obtained. The Tg value measured was 190° C.

### EXAMPLE 2

Production of N,N′-diphenyl-N,N′-bis-(4-triphenyl-methyl-phenyl)-amino-9-methyl-carbazol (formula 10)

In an apparatus as described in Example 1, 20.35 g 2,7-dianilino-9-methyl-carbazol and 49.4 g 4-bromophenyl-tri(-4-methylphenyl)-methane are reacted according to the procedure indicated in that same example using 12.9 g sodium-tert.-butylate as dehydrating base, 12.6 mg palladium acetate and 10.4 ml of a 1 % solution of tri-tert.-butylphosphine as catalyst.

The separation, processing and cleaning of the reaction product are carried out analogously to Example 1 too. In this way, approx. 17 g pure N,N′-diphenyl-amino-N,N′-bis-(4-(tri-4-methylphenyl)-methyl)-phenylamino-9-methyl-carbazol is obtained. The Tg value measured using a DSC measuring device is 159° C.

### EXAMPLE 3

Production of N,N′-di-(triphenylsilyl-phenyl)-N,N′-diphenyl-benzidine (formula 7)

In an apparatus as described in Example 1, 14.2 g N,N′-diphenyl-benzidine and 34.9 g 4-bromophenyl-triphenyl-silane are reacted according to the procedure indicated in that same example using 12.9 g sodium-tert.-butylate as dehydrating base, 12.6 mg palladium acetate and 10.4 ml of a 1% solution of tri-tert.-butyl-phosphine as catalyst.

The reaction product is cleaned by means of re-crystallization out of xylol to which 5% silica gel have been added, and, in a second stage, by re-crystallization out of DMF. In this way, 16.5 g pure N,N′-di-(triphenylsilyl-phenyl)-N,N′-diphenyl-benzidine is obtained, whose glass transition temperature measured using DSC is 164° C.

### EXAMPLE 4

Production of N-4-methylphenyl-N-(triphenylmethyl-phenyl)-N′-phenyl-N′-napth-1-yl-p,p′-benzidine (formula 12)

In the apparatus described in the above examples, 18.9 g bromobiphenylyl-phenyl-naphthyl-amine and 17.9 g trityl-methyl-diphenylamine are reacted in an analogous manner using 12.9 g sodium-tert.-butylate as dehydrating base, 12.6 mg palladium acetate and 10.4 ml of a 1 % solution of tri-tert.-butylphosphine as catalyst.

The reaction product is cleaned analogously to Example 1, wherein in a first stage a solvent mixture consisting of dodecane and xylol in a ratio of 4:1 and in a second stage a mixture of DMF and n-butanol in a ratio of 1:1 is used.

In this way, 20 g N-4-methylphenyl-N-(triphenylmethyl-phenyl)-N′-phenyl-N′-napth-1-yl-p,p′-benzidine is obtained. The glass transition temperature of the aforesaid compound is 151° C.

### EXAMPLE 5

Production of N,N′-bis-(-7-(N-(4-triphenylmethyl-phenyl)-N-phenyl-amino)-dibenzo-thiophene-2-yl)-N,N′-diphenyl-benzidine (formula 21)

In the apparatus described above, 36.1 g N,N′-bis-(-7-bromo-dibenzo-thiophene-2-yl)-N,N′-diphenyl-benzidine are reacted with 34.6 g N-tritylphenyl-N-phenyl-amine. The compounds indicated in Example 1 are used as catalysts in the amounts indicated in that same example. The product is precipitated using methanol after a reaction time of 7 hours.

The raw product is cleaned by means of recrystallization out of xylol and by recrystallizing it out of DMF three times. In this way, 22 g N,N′-bis-(-7-(N-(4-tri-phenylmethyl-phenyl)-N-phenyl-amino)-dibenzothiophene-2-yl)-N,N′-diphenyl-benzidine is obtained whose glass transition temperature is 186° C.

### EXAMPLE 6

Electroluminescent Arrangement

Under an ultra-high vacuum (10⁻⁸ hPa), a coating is applied onto a glass substrate coated with an indium tin oxide electrode (ITO). The aforesaid coating consists of a 55 nm thick hole transport layer consisting of the known starburst compound 25, 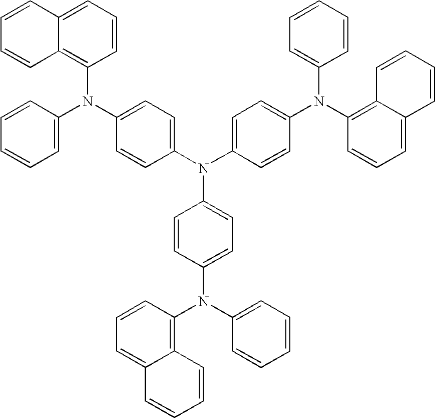
a 5 nm thick emissive layer consisting of N,N′-bis-(4′-(N-triphenylmethyl)-phenyl)-N-naphth-1-yl-amino)-biphenylyl)-N,N′-bisphenyl-2,7-amino-N-phenylcarbazol as obtained according to Example 1, and a 30 nm thick electron transfer layer consisting of the AIQ₃ chelate complex. The layers are precipitated at a growth rate of approx. 0.1 nm/s. Subsequently, a 90 nm thick aluminium cathode is applied onto the aforesaid structure.

A voltage is applied between the ITO electrode and the aluminium cathode in order to determine the electroluminescence curve. The luminous efficiency is measured using a large-area Si photodiode which is arranged immediately below the glass substrate.

The following results were achieved:

| | | |
|---|---|---|
| Turn-on voltage (1 cd/m²) | 2.8 | volts |
| Max. luminance (15 V) | 31,200 | cd/m² |
| Photometric efficiency (100 cd/m²) | 2.40 | cd/A |
| Lum. efficiency (100 cd/m²) | 1.20 | cd/W |

| | |
|---|---|
| Ext. quantum efficiency | 0.52% |

### EXAMPLE 7

Electroluminescent Arrangement

The same arrangement of layers is produced as in Example 6, except that N,N′-diphenyl-N,N′-bis-(4-triphenyl-methyl-phenyl)-amino-9-methyl-carbazol according to Example 2 is used in the emissive layer.

The following results are achieved:

| | | |
|---|---|---|
| Turn-on voltage (1 cd/m²) | 2.9 | volts |
| Max. luminance (15 V) | 24,100 | cd/m² |
| Photometric efficiency (100 cd/m²) | 2.15 | cd/A |
| Lum. efficiency (100 cd/m²) | 1.28 | cd/W |

| | |
|---|---|
| Ext. quantum efficiency | 0.39% |

The above examples show that substances produced according to the invention have glass transition temperatures of more than 150° C. In addition, the aforesaid substances showed an extremely low tendency to recrystallize in the amorphous layers whose production they were used for.

The invention has been described with reference to various specific and illustrative embodiments and techniques. However, it should be understood that many variations and modifications may be made while remaining within the spirit andscope of the invention.

1. A triarylamine compound having a general formula 1 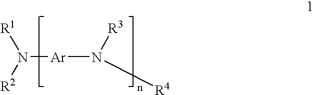
wherein
n is an integer from 1 to 10;
R¹, R², R³ and R⁴ are the same or different and are phenyl, biphenylyl, naphthyl, phenanthrenyl, anthracenyl, fluorenyl, triarylmethyl-aryl or triarylsilyl-aryl, which are optionally substituted by one or more of substituents C₁ to C₃ alkyl, C₁ to C₂ alkoxy and halogen; and at least one of the substituents R¹ through R⁴ being triarylmethyl-aryl or tri-arylsilyl-aryl according to formula 4 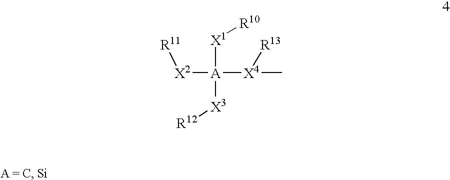
where the aromatic or heteroaromatic units X¹ through X⁴ are the same or different and are phenyl, naphthyl, anthracenyl, phenanthrenyl, pyrenyl, pyridyl or chinolyl; wherein R¹⁰, R¹¹, R¹² and R¹³ are the same or different amd are H, C₁ to C₆ alkyl, cycloalkyl, C₂ to C₄ alkenyl, C₁ to C₄ alkoxy, C₁ to C₄ dialkylamino, diarylamino, halogen, hydroxy, phenyl, naphthyl or pyridyl;
Ar is a structure according to formula 2 or 3 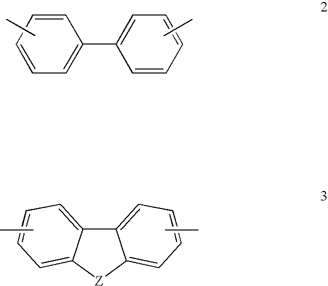
wherein the Ar are the same or different if n>1; and
where Z in formula 3 is selected from among the following structures 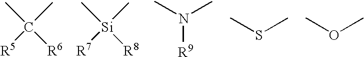
wherein R⁵ through R⁹ are the same or different and are H or C₁ to C₁₅ alkyl, or R₅ and R₆ or R₇ and R₈ combine to form a 5-membered or 6-membered alicyclic or heterocyclic ring thereby forming a spiro ring system together with the five-membered ring they are bonded to, wherein 0, N or S can be the heterocyclic elements;
or Ar is a structure according to formula 29, 30, 31 or 32 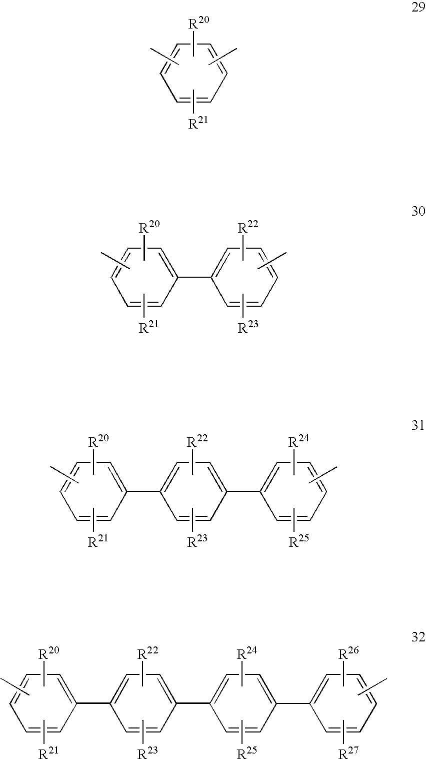
and where R²⁰ through R²⁷ are the same or different and are H, phenyl, C₁ to C₅ alkyl or C₁ to C₃ alkoxy and Ar is connected to the adjacent nitrogen atoms in any free substitution position;
with the proviso that, if n=1 or 2 and Ar is biphenylene or one of the groups according to formulas 29 through 32, at least one of the substituents R¹ through R⁴ is a triarylsilylaryl group, a substituted triarylsilylaryl group, or a substituted triarylmethylaryl group according to the above formula 4, wherein R¹⁰ through R¹² have the meaning specified above.

2. The triarylamine compound of claim 1, wherein n is 1, 2, 3 or 4.

3. The triarylamine compound of claim 1, wherein the substituents R¹ through R⁴ are the same or different and are phenyl, biphenylyl, naphthyl, fluorenyl, tri-(methylaryl)methylaryl or triarylsilylaryl.

4. The triarylamine compound of claim 1, wherein the substituents R⁵ through R⁹ are the same or different and are methyl or phenyl.

5. The triarylamine compound of claim 1, wherein the substituents R⁵ and R⁶ form a cycloalkane ring together with the C atom they are bonded to.

6. The triarylamine compound of claim 1, wherein the substituents R²⁰ through R²⁷ are the same or different and are H, methyl or phenyl.

7. The triarylamine compound of claim 1 having a general formula 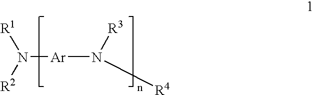
wherein
n is an integer from 1 to 10;
R¹, R², R³ and R⁴ are the same or different and are phenyl, biphenyl, naphthyl, tri-phenylmethylphenyl or triphenylsilylphenyl, which are optionally substituted by one or more of substituents C₁ to C₃ alkyl, C₁ to C₂ alkoxy and halogen; and
at least one of the substituents R¹ through R⁴ is a phenyl group substituted with a triphenylsilyl group or a substituted triphenylmethyl group having formula 33 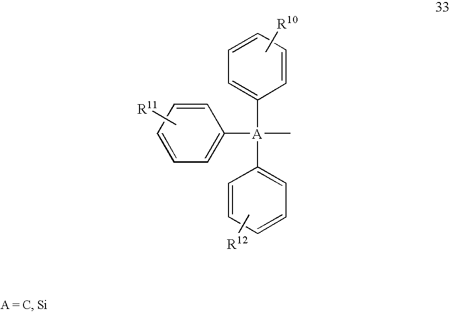
wherein R¹⁰, R¹¹ and R¹² are the same or different and are H, C₁ to C₆ alkyl, cycloalkyl, C₂ to C₄ alkenyl, C₁ to C₄ alkoxy or halogen,
and where R¹ through R⁴ can be substituted by one or more substituent(s);
Ar is 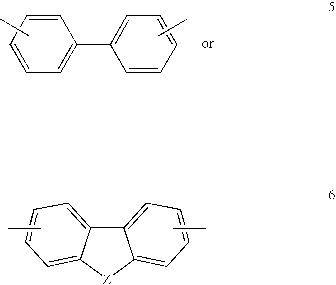
wherein Z is selected from among the following structures 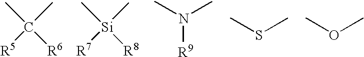
and R⁵ through R⁹ are the same or different and are H or C₁ to C₅ alkyl, on condition that, if n=1 and Ar is biphenyl, at least one of the substituents R¹ through R⁴ is a phenyl group substituted with a triphenylsilylphenyl substituent according to the above formula 33, wherein R¹⁰ through R¹² have the meaning specified above.

8. An organic electroluminescent device comprising at least one hole transport layer and one luminescent layer, wherein at least one hole transport layer includes a triarylamine compound according to claim 1.

9. The organic electroluminescent device of claim 8, wherein at least one luminescent layer includes the triarylamine compound.

10. The organic electroluminescent device of claim 8, comprising the triarylamine compound as hole transfer substance in an electrophotographic arrangement.

11. A triarylamine compound having a general formula 1 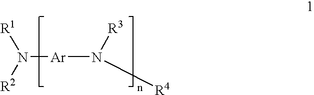
wherein n is 1; and
R¹, R², R³ and R⁴ are the same or different and are phenyl, biphenylyl, phenyl, naphthyl, triarylmethyl-aryl or triarylsilyl-aryl, which are optionally substituted by one or more of C₁ to C₃ alkyl, C₁ to C₂ alkoxy and halogen; and at least one of the substituents R¹ through R⁴ is triarylsilyl-aryl according to formula 4 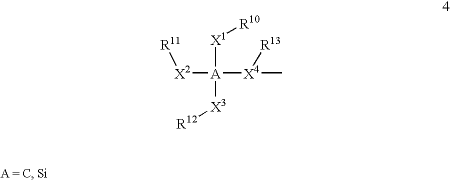
wherein the aromatic groups X¹ through X⁴ are the same or different and are phenyl or naphthyl; wherein R¹⁰, R¹¹, R¹² and R¹³ are the same or different and are H, C₁ to C₆ alkyl, cycloalkyl, C₂ to C₄ alkenyl, C₁ to C₄ alkoxy, C₁ to C₄ dialkylamino, or halogen; and
Ar is 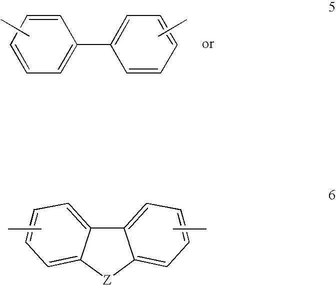
where Z is selected from among the following structures 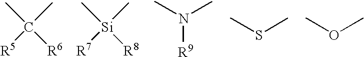
wherein R⁵ through R⁹ are the same or different and are H or C₁ to C₁₅ alkyl, or R₅ and R₆ or R₇ and R₈ combine to form a 5-membered or 6-membered alicyclic or heterocyclic ring thereby forming a spiro ring system together with the five-membered ring they are bonded to, wherein O, N or S can be the heterocyclic elements.

12. The triarylamine compound of claim 11, wherein the substituents R⁵ through R⁹ are the same or different and are methyl or phenyl.

13. The triarylamine compound of claim 11, wherein the Ar is

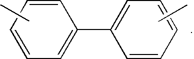

14. A triarylamine compound having a general formula 1 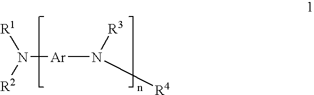
wherein n is 1; and
R¹, R², R³ and R⁴ are the same or different and are phenyl, biphenylyl, naphthyl, triarylmethyl-aryl or triarylsilyl-aryl, which are optionally substituted by one or more of C₁ to C₃ alkyl, C₁ to C₂ alkoxy and halogen; and at least one of the substituents R¹ through R⁴ is a triphenylmethylphenyl or triphenylsilylphenyl group according to formula 4 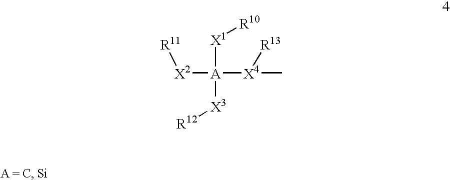
wherein the aromatic groups X¹ through X⁴ are the same or different and are phenyl or naphthyl; wherein R¹⁰, R¹¹, R¹² and R¹³ are the same or different and are C₁ to C₆ alkyl, cycloalkyl, C₂ to C₄ alkenyl, C₁ to C₄ alkoxy, C₁ to C₄ dialkylamino, or halogen; and
Ar is 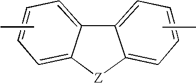
where Z is selected from among the following structures 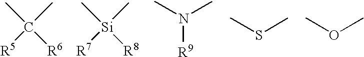
wherein R⁵ through R⁹ are the same or different and are H or C₁ to C₁₅ alkyl, or R₅ and R₆ or R₇ and R₈ combine to form a 5-membered or 6-membered alicyclic or heterocyclic ring thereby forming a spiro ring system together with the five-membered ring they are bonded to, wherein O, N or S can be the heterocyclic elements.

15. The triarylamine compound of claim 14, wherein Z is —O—.

16. The triarylamine compound of claim 14, wherein Z is —S—.

17. The triarylamine compound of claim 14, wherein Z is —N(Rg)—.

18. The triarylamine compound of claim 14, wherein Z is —Si R₆(R₅)—.

19. The triarylamine compound of claim 14, wherein Z is —C R₆(R₅)—.

20. The triarylamine compound of claim 14, wherein A is C.

21. The triarylamine compound of claim 20, wherein X¹ through X⁴ are phenyl.

22. The triarylamine compound of claim 14, wherein A is Si.

23. The triarylamine compound of claim 22, wherein X¹ through X⁴ are phenyl.

24. A triarylamine compound having a general formula 1 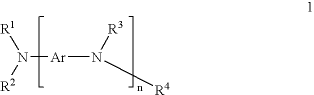
wherein n is 1;
Ar is 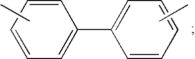
and
R¹, R², R³ and R⁴ are the same or different and are phenyl, biphenylyl, phenyl, naphthyl, triarylmethyl-aryl or triarylsilyl-aryl, which are optionally substituted by one or more of C₁ to C₃ alkyl, C₁ to C₂ alkoxy and halogen; and at least one of the substituents R¹ through R⁴ is a triphenylsilylphenyl group or a substituted triphenylmethyl group according to formula 4 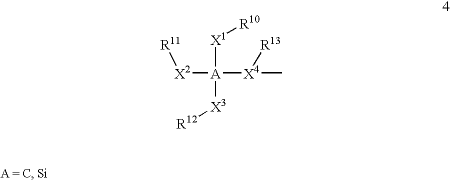
wherein the aromatic groups X¹ through X⁴ are the same or different and are phenyl or naphthyl; wherein R¹⁰, R¹¹, R¹² and R¹³ are the same or different and are H, C₁ to C₆ alkyl, cycloalkyl, C₂ to C₄ alkenyl, C₁ to C₄ alkoxy, C₁ to C₄ dialkylamino, or halogen; and at least one of the substituents R¹ through R⁴is a substituted triarylmethylaryl group according to the above formula 4.

25. The triarylamine compound of claim 24, wherein A is Si.

26. The triarylamine compound of claim 25, wherein X¹ through X⁴ are phenyl.
